(19) United States
(12) Patent Application Publication
(10) Pub. No.: US 2017/0173225 A1
(43) Pub. Date: Jun. 22, 2017

(54) METHODS FOR COATING IMPLANT SURFACES TO TREAT SURGICAL INFECTIONS

(71) Applicant: BIOMET MANUFACTURING, LLC, Warsaw, IN (US)

(72) Inventor: Karen S. Troxel, Warsaw, IN (US)

(21) Appl. No.: 15/302,119

(22) PCT Filed: Apr. 16, 2015
(86) PCT No.: PCT/US15/26157

§ 371 Date: Oct. 5, 2016

Related U.S. Application Data
(60) Provisional application No. 61/980,406, filed on Apr. 16, 2014.

(51) Int. Cl. A61L 31/16; A61L 31/14; A61K 31/496; A61B 17/68; A61K 38/14; A61K 31/7036; A61F 2/30; A61L 31/08; A61K 31/65

(52) U.S. Cl. CPC A61L 31/16; A61L 31/08; A61L 31/146; A61K 31/496; A61K 31/65; A61K 38/14; A61K 31/7036; A61F 2/30771; A61B 17/68; A61L 2300/404; A61L 2300/406; A61L 2300/22; A61F 2310/0097; A61F 2002/3069; A61F 2002/3092; A61F 2002/30878; A61F 2002/30827; A61B 2017/00889

(57) ABSTRACT

Methods for treating infection at the site of implantation of an orthopedic device in a human or animal subject. The methods include removing the orthopedic device, and implanting a replacement device. A surface of the replacement device is coated with an infection-inhibiting composition having a waxy matrix. The waxy matrix includes an infection-inhibiting material, such as a lipid, an antimicrobial agent, or a combination of a lipid and an antimicrobial agent.

### INTRODUCTION

The present technology relates to orthopedic implants with an infection-inhibiting agent, methods for making orthopedic implants with an infection-inhibiting agent, and methods for treating infection at the site of implantation of an orthopedic device in a human or animal subject.

Orthopedic implants are implantable medical devices used to replace, augment or repair bone, such as to replace diseased articulating joints (such as knees, hips and elbows), stabilize the skeleton where it has been destabilized by trauma (such as fractures), or to correct alignment. These implants are manufactured most commonly with plastics, polymers, ceramics, steel, stainless steel, metals and alloys.

However, as foreign bodies, the surfaces of orthopedic devices implanted into the body provide a physical platform for bacteria to attach and grow. Due to the rapid growth rate and presence of virulence factors, bacteria are able to establish infections within days of the surgical procedure causing loss of implant fixation, local tissue inflammation, and local tissue necrosis due to sepsis. The most common organisms causing these infectious complications are Staphylococcus epidermidis, Staphylococcus aureus and Pseudomonas sp. when the injuries are related to trauma or battlefield injuries. In the case of orthopedic procedures, Staphylococcus epidermidis, Staphylococcus aureus account for almost 70-80% of all infectious organisms, with Staphylococcus epidermidis being the most common organism.

Such device-related infections may not be successfully treated by systemic antibiotics alone. Currently, the treatment for infected joint prostheses in a subject includes either a two-stage procedure or revision or a one-stage procedure or revision. A two-stage revision includes a first surgery for removing implanted hardware, and implanting an antibiotic loaded bone cement spacer. The spacer is left in the subject for about six weeks while the subject concurrently receives six weeks of systemic antibiotic administration. After the infection is cleared, a second surgery is performed in which the spacer is removed and the final joint replacement hardware is implanted, with or without cement. In contrast, a one-stage revision includes a single surgery for removing the infected hardware, debriding the implantation site, and implanting new hardware with antibiotic-loaded cement. One-stage revisions also typically include systemic antibiotic administration over a course of time.

There is significant morbidity associated with the two-stage revision, and even though it may involve extensive hospitalization and multiple surgeries, it is more common than one-stage revisions because of a perception that it is more successful in curing infections. One-stage revisions are associated with less morbidity and cost, relative to two-stage revisions, but are used less frequently because a cemented prosthesis is much harder to remove than an uncemented prosthesis. Because the “cure” rate associated with a one-stage procedure is typically between 60-80%, there is a significant probability that reinfection will occur, requiring yet another revision surgery.

Accordingly, there is a need for a one-stage uncemented revision joint prosthesis. The prosthesis would be easier to revise than a cemented prosthesis if reinfection occurs. Moreover, such a prosthesis would improve the current standard of care by avoiding a significant amount of morbidity and cost relative to a two-stage revision procedure. Such an uncemented joint prosthesis would also be useful during an initial joint repair procedure.

### SUMMARY

The present technology provides infection-inhibiting compositions suitable for coating surfaces of implantable medical implants, including compositions and devices for coating medical devices in the operating room prior to implantation in a patient. The compositions have a waxy matrix comprising an infection-inhibiting material, and are operable to deliver the infection-inhibiting material to the surface of an implant. In some embodiments, such compositions comprise a waxy material comprising a lipid as an infection-inhibiting material, wherein the waxy material is operable to deposit the lipid when rubbed on a surface of a device. Lipids useful herein include long-chain diacylglycerides or triacylglycerides, which may be saturated or unsaturated. A preferred lipid comprises a phospholipid, such as lecithin or a purified form of phosphatidylcholine.

The present technology also provides methods for treating infection at the site of implantation of an orthopedic device in a human or animal subject. The method includes removing the device; and implanting a replacement device. A surface of the replacement device is coated with an infection-inhibiting composition having a waxy matrix. The matrix can comprise an infection-inhibiting material selected from the group consisting of a lipid, an antimicrobial agent, and mixtures thereof.

Also provided by the present technology are methods for treating infection at the site of implantation of an orthopedic device in a human or animal subject, comprising removing the device; rubbing a surface of a replacement device with an infection-inhibiting composition having a waxy matrix, the matrix comprising an infection-inhibiting material selected from the group consisting of a lipid, an antimicrobial agent, and mixtures thereof; and implanting the replacement device.

Methods for treating infection at the site of implantation of an implanted orthopedic device in a human or animal subject are also provided by the present technology. The methods comprise:
(a) obtaining a replacement device having a textured surface coated with an infection-inhibiting composition having a waxy matrix, the matrix comprising a lipid, an antimicrobial agent, and mixtures thereof, wherein the antimicrobial agent is selected based on a diagnostic assessment of the infection;
(b) removing the implanted device; and
(c) implanting a replacement device.

Additionally, the present technology provides methods for making replacement implants for use in a revision procedure in a subject having an infection at the site of an implanted device. The methods comprise obtaining a three-dimensional image of the bone at the site; manufacturing the replacement implant having a textured surface using the three-dimensional image; and coating the surface with an infection-inhibiting composition having a waxy matrix, the matrix comprising a lipid, an antimicrobial agent, and mixtures thereof.

### DRAWINGS

FIG. 1 (FIGS. 1A-1C) is a diagrammatic illustration of an infection-inhibiting delivery system suitable for coating an implant.

It should be noted that the FIGURE set forth herein is intended to exemplify the general characteristics of materials and methods among those of the present technology, for the purpose of the description of certain embodiments. This FIGURE may not precisely reflect the characteristics of any given embodiment, and is not necessarily intended to define or limit specific embodiments within the scope of this technology.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. A non-limiting discussion of terms and phrases intended to aid understanding of the present technology is provided at the end of this Detailed Description.

The present technology provides compositions, methods and systems for pre-operative or intra-operative coating of medical implants (e.g., orthopedic implants) with an infection-inhibiting material. Compositions comprise a waxy matrix comprising an infection-inhibiting material.

### Waxy Matrix

The waxy matrices of the compositions of the present technology are operable to deposit an infection-inhibiting material when rubbed on a surface of a device. The matrix can be formulated into a stick that holds its shape under gentle pressure used to transfer the matrix onto a surface of an implant. In particular, as discussed further below, the composition is solid and does not readily flow (except under force).

Waxy matrices useful preferably herein include those that 1) leave a thin layer on the surface of an implant when rubbed over its surface; 2) dissolve, resorb or otherwise dissipate from the implant surface after implantation; and 3) do not illicit any adverse tissue reactions at the locus of the implant or after absorption into the blood or lymphatic system. Further, in compositions comprising an optional antimicrobial agent, the waxy matrices are also mixable with antimicrobial agent at temperatures below temperatures that lead to thermal destruction of the antimicrobial agents. Preferably the waxy matrix does not interfere with tissue adhesion to the surface of the device, especially for implants such as non-cemented joint replacement components or dental implants. Preferably the compositions of the present technology are substantially free of solvents.

In various embodiments, the matrix comprises, or consists essentially of, lipids. As discussed below, the matrix may comprise a lipid as an infection-inhibiting material. Without limiting their mechanism, function, or utility, such compositions are biocompatible, do not interfere with bone repair, dissolve readily in vivo, and are metabolized by the body leaving nontoxic degradation products. Compositions can comprise lipids that are in solid phase at room temperature and that either naturally have a waxy consistency or have been formulated with other materials, such as other lipids and viscosity modifiers, to produce a waxy consistency.

Lipids useful herein include naturally occurring compounds that generally are defined as fatty acids and their derivatives, and biosynthetically or functionally related compounds. Classes of lipid structures include fatty acids, ecosanids, simple glycerolipids, sterols and other isoprenoid lipids, lipoproteins, complex glycerolipids including glycerophospholipids and glycosyldiacylglyerols, and sphingolipids. Preferred lipids include fatty acids, simple glycerolipids including triacylglyerols and diacylglycerols, and glycerophospholipids (phospholipids). The lipid-based composition may comprise sufficient long-chain diacylglycerides or triacylglycerides, either saturated or unsaturated, to provide stiffness to the formulation so that it is solid at room temperature and has a waxy texture. Examples of saturated fatty acids include caproic acid, enanthic acid, caprylic acid, pelargonic acid, capric acid, undecylic acid, lauric acid, tridecylic acid, myristic acid, pentadecylic acid, palmitic acid, margaric acid, stearic acid, nonadecylic acid, arachidic acid, heneicosylic acid, behenic acid, tricosylic acid, lignoceric acid, pentacoylic acid, cerotic acid, heptacosylic acid, montanic acid, nonacosylic acid, melissic acid, nenatriacontylic acid, lacceroic acid, psyllic acid, geddic acid, ceroplastic acid and hexatriacontylic acid. Examples of unsaturated fatty acids include myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid and docosahexaenoic acid. Furthermore, the unsaturated fatty acids may be polyunsaturated. In addition to the lipids that are solid at room temperature, other lipids that are liquid at room temperature may be added to soften the hardness of the solid lipids.

In some embodiments, the lipid matrix comprises (by weight % of the matrix) about 50% or more, about 60% or more, 70% or more, 80% or more, 90% or more, or 95% or more phospholipid. In some embodiments, the lipid matrix consists essentially of phospholipids. Phospholipids are amphipathic molecules that are characterized by a hydrophilic head, which consists of a polar group, a phosphate and glycerol, and two hydrophobic fatty acid tails, which may be saturated, unsaturated, polyunsaturated or combinations thereof. The fatty acids of synthetic phospholipids may be medium-chain fatty acids with 6-12 carbons, long-chain fatty acids with more than 12 carbons or very long-chain fatty acids with more than 22 carbons. In various embodiments, the fatty acid tails are either 16 or 18 carbons long, wherein the 18-carbon chains are predominantly unsaturated.

In some embodiments, the phospholipid is a mixture of phospholipids, for example, a mixture of phosphatidylcholine, phosphatidylethanolamine and phosphatidylinositol as is present in lecithin. Lecithin may be isolated from plant (such as soy bean) and animal tissues. Commercially available lecithin useful herein includes Phosal® 53 MCT, (from Lipoid, GmbH, Köln, Germany) which comprises at least about 53% phosphatidylcholine, up to 6% lysophosphatidylcholine and from 3% to 6% ethanol.

Phosphatidylcholine is a preferred phospholipid. Phosphatidylcholines are the most abundant form of lipid component in cellular membranes. They are amphipathic, having both hydrophobic sections in the fatty acid tails and hydrophilic portions in the phosphate and choline groups. In naturally occurring phosphatidylcholines the fatty acid tails are either 16- or 18-carbons long and the 18-carbon chains are predominantly unsaturated. Such a preparation is commercially available as Phospholipon® 90G (from Lipoid GmbH, Köln, Germany) and comprises at least 94% phosphatidylcholine, up to 4.0% lysophosphatidylcholine and up to 0.3% tocopherol. Phosphatidylcholine derived from soybeans and purified is a slightly yellow, waxy solid. Purified phosphatidylcholine such as Phospholipon 90G makes an excellent coating stick and it is also an excellent carrier for optional additives such as bioactive antimicrobial agents. Purified phosphatidylcholine from either soybean or from eggs is available from several commercial sources; it is used in the food processing industry. Other glycerophospholipids such as phosphatidylserine, phosphatidylinositol, and phosphatidylethanolamine may be used.

Synthetic phosphatidylcholines are also available from several sources and with different lengths of fatty acid chains and different degrees of saturation. These are readily available commercially and are used in drug delivery formulation. Also, naturally derived purified phosphatidylcholine can be hydrogenated to fully saturate the fatty acid chains, producing a solid that is a white powder and no longer has a waxy consistency. The hydrogenated phosphatidylcholine and the synthetic phosphatidylcholines can be combined with other lipids to create a stick product with the physical characteristics necessary to achieve the proper hardness and the ability to transfer easily and smoothly to the surface of a device. For instance, hydrogenated phosphatidylcholine can be added to natural unsaturated phosphatidylcholine in order to increase the stiffness of the stick product. Alternatively, lipids that are liquid at room temperature such as medium chain triacylglycerols found in corn oil, olive oil, palm oil, sunflower oil, or rapeseed oil for instance, or unsaturated simple fatty acids such as oleic or linolenic acid, can be used to soften a stiffer lipid such as hydrogenated phosphatidylcholine or fully saturated triacylglycerols such as tristearate, trimyristate, or tripalmitate in order to produce a coating stick product with the proper hardness and ease of application. If an additive such as an antimicrobial agent needs to be pre-mixed with a liquid lipid mixture such as Phosal 53MCT prior to addition to the matrix in order to utilize a mechanical mixing process, then a stiffer lipid such as a fully saturated (hydrogenated) phosphatidylcholine can be added to the coating stick formulation in order to counteract the softening effects of the liquid lipid component.

For application to the surfaces of orthopedic implants that require biologic fixation, purified soy derived phosphatidylcholine is a particularly preferred lipid because it dissolves readily and will not interfere with biologic fixation, and is biocompatible and compatible with bone repair. The amphipathic nature and the unsaturated fatty acid components allow the phosphatidylcholine to disperse rapidly in aqueous environments such as in vivo. Fully saturated triacylglyerols and saturated fatty acids are not readily soluble in water, hindering dispersion in vivo. This may limit their application to the surface of implants that do not require bone or tissue attachment, such as fracture hardware, plates, screws, intramedullary nails. Accordingly, except as used to modify the rheology of compositions as discussed above, the compositions of the present technology are preferably substantially free (containing less than 5%, preferably less than 1%, or preferably about 0%) of fully saturated triacylglyerols and saturated fatty acids.

As discussed above, the compositions of this technology have a rheology that is a non-flowable solid at ambient conditions, preferably also at body temperature. In some embodiments, the compositions are not “putty-like” or malleable with moderate pressure (by hand) under ambient conditions. In some embodiments, the compositions have a consistency between that of beeswax and semi-flowable putty. The physical properties of a composition renders it operable to transfer material from the composition, in particular an infection-inhibiting material, onto a surface of an implant by rubbing the composition over the surface with moderate pressure, by hand. The stiffest formulations can be applied to the surface of a device by direct application, such like coloring with a crayon. A more malleable formulation can be applied to the surface of a device with hand pressure or smearing, or with a spatula or similar applicator. Preferably the resulting coating is smooth, even, adherent, not flaky, and easily applied. The coating may transfer to tissue at the site of implantation, while remaining sufficiently pliable and adherent that it does not come off of the surface of the implant in flakes. Such compositions may have a viscosity equivalent to crayons, lipstick, lip balm, and similar compositions known in the art for the delivery of pigments, cosmetic and pharmaceutical actives to tissues and other surfaces.

The composition preferably has a Cone Penetration Hardness of from at least about 1.5 lbf to about 15 lbf in a cone penetration test, as described below. “Cone Penetration Hardness” is defined as the peak force experienced in moving a penetration cone through the composition for a distance of 5 mm at a rate of 1 mm/second. The cone has a maximum diameter of 6.25 mm and widens from a point to a maximum diameter over a length of 6 mm at an angle of 27.5 degrees. In various embodiments, the hardness is at least 3 lbf, at least about 5 lbf, or at least about 10 lbf. The hardness may be less than about 10 lbf, in some embodiments.

### Infection-Inhibiting Material

Compositions of the present technology comprise a safe and effective amount of an infection-inhibiting material in a waxy carrier operable to deposit the anti-infective when rubbed on a surface of the device. Such a “safe and effective amount” is sufficient to have the desired infection-inhibiting effect in the human or lower animal subject, without undue adverse side effects (such as toxicity, irritation, or allergic response), commensurate with a reasonable benefit/risk ratio when used in the manner of this technology. The specific safe and effective amount of the infection-inhibiting material will vary with such factors as the particular surgical procedure, the surface characteristics of the implant device (such as material, texture and contours), the condition and characteristics of the tissue into which the device is implanted the physical condition of the patient, the nature of concurrent therapy (if any), the specific infection-inhibiting material used, and other materials (if any) present in the composition matrix. The infection-inhibiting effect preferably substantially reduces the number of microbes on the treated surface of the implant after implantation of the device relative to the number of microbes that would be present on the implant without coating.

As referred to herein, an “infection-inhibiting” material inhibits the attachment of one or more microbial organisms (e.g., bacteria, yeast and other fungal organisms) on the surface to which it is applied. Such inhibition of surface attachment prevents development of biofilm-based microbial phases of growth at the site of the implant, thus the prevention of adhesion maintains the microbes in a state bioavailable to the host immune system. In some embodiments, microbial growth is substantially prevented or suppressed, wherein microbes are present (if present) following implantation at a level allowing the immune system of the surgical subject to recognize and neutralize remaining microbes. The infection-inhibiting material reduces the likelihood of device-related infection or surgical site infection. In various embodiments, a subject with an infected joint implant may be treated with a one stage revision procedure in which the infection-inhibiting material in the waxy carrier is coated on an uncemented revision implant. Such a treatment may be accompanied with a systemic administration of antibiotics for a period of time. Therefore, the infection-inhibiting material may also be referred to as an “infection-treating” material.

In orthopedic surgical procedures, microbes (“target microbes”) include organisms that are associated with the device or may be otherwise present at the site of the device, which may in bone or surrounding tissues such as skin, blood, muscle, cartilage, and bone. Thus, any microbe that has the potential to enter surreptitiously and colonize at a surgical site or area of orthopedic repair and trauma may be targeted in accordance with the present technology. Target microbes of particular concern are those that colonize the skin of a surgical subject, since these organisms may enter the subject at the site where the orthopedic implant was inserted.

Particularly relevant target microbes include Gram-positive and Gram-negative bacteria, and yeasts. Such organisms include Klebsiella, Enterobacter, Acinetobacter, Pseudomonas, Escherichia, and Staphylococcus. Specific bacteria include Staphylococcus aureus, as represented by strain NCTC 8325 and methicillin resistant strains which presently cause significant problems in hospital environments. Further targets are Staphylococcus epidermidis, represented by strain NCTC 11047, and yeasts such as Candida albicans, represented by strain ATCC 26555. Some of these bacteria are known to produce fibrinogen-binding clumping factors A and B and the fibronectin-binding protein (FnbA), capable of adhering to orthopedic implants and related devices.

As discussed above, the infection-inhibiting material may comprise a lipid, which may be a lipid constituting, in whole or in part, the waxy matrix. Thus, in some embodiments, compositions of the present technology consist essentially of an infection-inhibiting lipid, wherein the composition does not contain an antimicrobial agent (as defined and exemplified below). Without limiting the mechanism, function or utility of the compositions and methods of this technology, in some embodiments the compositions comprising a lipid inhibit attachment of organisms to the surface of the implant onto which they are applied, such as by providing a temporary physical barrier to adhesion of bacteria on the surface of the implant. In this regard, it is generally understood that the attachment of bacteria and other microbes to the surface of a device is the first step in the progression of events leading to device-related infection. Subsequent steps involve propagation and creation of a microbial community protected by a polysaccharide extracellular structure known as “biofilm.” When attached to the device surface, the bacteria become less available to the host immune system and in biofilm structures they also become less susceptible to the antimicrobial effects of antibiotics. The inhibition of attachment by the compositions of this technology thus keeps the bacteria available to the host immune cells including white blood cells such as polymorphic neutrophils and macrophages.

The anti-adhesion effects of the lipid material may be extended or amplified by the addition of antimicrobial agents that inhibit the growth of the bacteria, inhibit bacterial metabolism, or inhibit the formation of biofilm. Thus, the infection-inhibiting material may be selected from the group consisting of a lipid, antimicrobial agent, and combinations thereof. Specifically, in some embodiments, the present technology provides compositions for application to the surface of an implantable medical device, comprising an antimicrobial agent in a waxy matrix operable to deposit the anti-infective when rubbed on a surface of the device. As noted above, the compositions are preferably resorbable or otherwise dissolvable, such that the optional antimicrobial dissipates from the surface of the device after the device is implanted.

Antimicrobial agents useful in the compositions of the present technology include any compound that has inhibitory activity against the growth of microbes, preferably bacteria as discussed above. Preferably, the anti-infective is selected from the group consisting of antibiotics, antimicrobial peptides, antimicrobial peptide mimetics, disinfectants, antiseptics, antimicrobial metal ions, sugar alcohols, essential oils, salicylic acid, methyl salicylate, nitrous oxide, and mixtures thereof. The amount of antimicrobial agent in the uniform antimicrobial composition is preferably at least about 0.1%, at least about 1%, at least about 5% or at least about 10% of the composition. In various embodiments, the concentration is 50% or less, 40% or less, 30% or less, or 20% or less of the composition. For example, the concentration of antimicrobial agent may range from about 0.1% to about 40%, or from about 5% to about 35% of the composition.

Suitable antimicrobial agents may have at least one or more of the following properties: 1) the ability to prevent growth and/or replication and/or to kill pathogens which become associated with the orthopedic implant through their ability to bind to blood, muscle and osseous tissue; 2) possessing an acceptable side effect profile, including low toxicity and allergenicity for the intended human or animal subject to be treated; 3) acceptable efficacy at the site of implantation of the coated device, with limited development of microbial resistance; 4) acceptable miscibility or solubility with the carrier; and 5) stability in the coating when applied to the implant.

Antibiotics useful herein include, for example, rifamycins (such as rifampin), fosfomycin, fusidic acid, glycylcyclines, aminoglycosides, quinolones, glycopeptides, bismuth thiols, sulfonamides, trimethoprim, macrolides, oxazolidinones, β-lactams, lincosamides, chloramphenicol, gramicidins, polymyxins, lipodepsipeptides, bacitracins, tetracyclines (such as minocycline), penicillin, ampicillin, cefazolin, clindamycin, erythromycins, levofloxacin, vancomycin, gentamycin, and mixtures thereof. In one embodiment, the antimicrobial agent comprises a mixture of vancomycin and gentamycin. For example, a composition can comprise a waxy matrix and vancomycin at a concentration of from about 2% to about 10% by weight of the composition and gentamycin at a concentration of from about 2% to about 10% by weight of the composition. Various compositions, materials, and spacers comprising vancomycin and gentamycin are disclosed in Patent Application Publication No. 2013/0150979, Schindler et al., published Jun. 13, 2013.

Tetracycline antibiotics refer to a number of antibiotics of either natural, or semi-synthetic origin, derived from a system of four linearly annealed six-membered rings (1,4,4a,5,5a,6,11,12a-octahydronaphthacene) with a characteristic arrangement of double bonds. The tetracycline antibiotic can include one or more tetracyclines, and/or semi-synthetic tetracyclines such as doxycycline, oxytetracycline, demeclocycline, lymecycline, chlortetracycline, tigecycline and minocycline. A preferred tetracycline is minocycline or minocycline hydrochloride. The amount of tetracycline present in the infection-inhibiting coating can range from about 5 μg/cm² to about 1000 μg/cm², or from about 10 μg/cm² to about 800 μg/cm².

Rifamycin class of antibiotics is a subclass of antibiotics from the ansamycin family of antibiotics. The present antibiotic agent or agents can include one or more rifamycin antibiotics from the group rifamycin B, rifampin or rifampicin, rifabutin, rifapentine and rifaximin. Rifampin is commercially available as Rifadin and Rimactane from Sanofi-Aventis U.S. LLC. (Bridgewater, N.J., USA).

Antimicrobial peptides useful herein include, for example, host defense proteins, defensins, magainins, cathetlicidins, protegrins, lantibiotics, nisins, and synthetic mimics of host defense proteins such as cationic steroids. Antiseptics and disinfectants include, for example, chlorhexidine, polyhexanide, triclosan, and iodine-delivering formulas such as betadine or povidone-iodine. Metal ions include various formulations of silver that effectively release silver ions, including silver salts and silver nanoparticles, or copper salts and copper nanoparticles that release copper ions.

Food preservatives that would effectively inhibit microbial attachment or growth include, for example, epsilon polylysine, nisin, and various essential oils including oils from cinnamon, thyme, clove, lemon, lime, orange, and geranium or purified active antimicrobial ingredients from essential oils such as cinnamaldehyde, garnesol, carvacrol, and thymol.

Other antimicrobial agents useful herein include salicylic acid and its metabolite methyl salicylate, and sugar alcohols and polyols (such as xylitol and erythritol). Such sugar alcohols can have antimicrobial properties by preventing bacterial adhesion or bacterial biofilm formation. Polysaccharides, such as chitosan and alginate, are also useful herein.

### Optional Materials

Compositions of the present technology may comprise other materials that (for example) alter the physical characteristics of the compositions or provide therapeutic benefits. Examples include antioxidants, colorants, viscosity modifying agents, and therapeutic actives. For example, polysaccharides, such as carboxymethylcellulose, can be added to improve handling or physical properties. Antioxidants, such as vitamins E and/or C (as tocopherol acetate and ascorbic acid for instance), may also be present. The compositions may also comprise optional active materials, such as small molecule drugs, such as anesthetics (such as bupivacaine) to manage pain, or therapeutic actives including bone and tissue growth promoters and anti-inflammatories. Therapeutic actives among those useful herein include bisphosphonates, insulin mimetics (such as vanadium compounds, including vanadyl acetylacetonate), growth factors, and cytokines.

### Methods of Manufacturing Compositions

The compositions of the present technology may be made by any suitable process for making lipid compositions, including methods among those known in the art for forming soft solid lipid-containing compositions. In various methods, methods comprise cold forming the composition into a final product form (e.g., a stick). In some embodiments, the forming may be conducted using moderate heat, at a temperature below which the lipid material will degrade.

Mechanical grinding and kneading may be used to make a cohesive composition in a final desired form, e.g., a stick. Mechanical grinding can be performed on an industrial scale by using compounding extruders, such as co-rotating twin screw extruders. Such twin screw extruders are used in the pharmaceutical industry for granulating and compounding, and in the food processing industry for kneading. The screws of the extruders may be either co-rotating or counter-rotating. Additives, such as antimicrobial materials, antioxidants, and other materials, as discussed above, can be added to a hopper either as powders or premixed as a slurry or solution with a liquid lipid, such as Phosal 53MCT or other lipid formulation that is liquid at room temperature. Moderate heat, such as limited exposure to heat at a temperature of from about 40° C. to about 80° C., may aid the mixing process. The mixed and extruded formulations can be cold pressed, or pressed with mild heat, into a desired shape, for example, a cylindrical stick-form, to insert into a dispensing apparatus, as discussed below. If natural phosphatidylcholine or hydrogenated phosphatidylcholine is used, heat is preferably limited to mild temperatures because phospholipids are subject to thermal degradation, and so cannot be melted and then cooled in a mold or applicator. Hot melt extrusion is preferably not employed. An advantage provided by using a compounding or kneading extruder is that there is no introduction of an organic solvent that would later need to be removed from the formulation. The mechanical mixing works well with purified natural phosphatidylcholine, which has a waxy texture as is, and needs no additive to modify the physical properties to get a good stiff waxy stick product.

Alternatively, a lipid based matrix carrier and optional additives can be mixed by first dissolving in organic solvents, preferably a biocompatible solvent, to form true solutions, then mixing the solutions together. A “biocompatible solvent” is a solvent that elicits little or no toxic response in a human or other animal subject. Such solvents useful herein include alcohols, diglycerides, triglycerides, glycerols, polyethylene glycols, saturated or unsaturated free fatty acids (including short, medium and long chain fatty acids and mixtures thereof), tocopherols (such as vitamin E, including tocopherol acetate, alpha tocopherol and gamma tocopherol), and mixtures thereof. The solvents are then removed to leave a completely homogenous lipid-based solid formulation that can be cold molded into a suitable form (e.g., a stick-form) for insertion into an applicator. The solvent removal can be accomplished by either freeze-drying or by spray-drying.

For freeze-drying, the equipment and the solvent are carefully selected so that the eutectic point, or the transition from frozen to sublimation, and therefore the shelf temperature to maintain in the drying phase, is feasible and also that the condenser temperature can be achieved to pull the solvent out of the exhaust. The vacuum pump should be explosion proof. The solvent may alternatively be a mixture comprising organic solvent and water. Appropriate solvents include tertiary butanol, ethanol, isopropanol, acetonitrile and methanol.

Spray-drying may be employed to remove an organic solvent, whereby a solution comprising lipid matrix and additives in the organic solvent is atomized (such as by pressure or ultrasonics) into the top of a tall drying chamber. Very dry gas (such as compressed nitrogen, air, or argon) is also introduced into the top of the chamber. The solvent evaporates into the gas as atomized droplets fall to the bottom of the chamber. The product is collected in the bottom of the chamber and can be removed and cold molded into an appropriate shape before insertion in an applicator. The gas phase is exhausted at the bottom of the chamber as well and sent through a condenser or chiller to remove the solvent for reuse or disposal. Non-limiting examples of solvents used in spray drying operations include methanol, ethanol, toluene, hexane, acetone, ethylacetate, and dichloromethane.

Solvent-based processing can be advantageous if the waxy matrix contains saturated lipids that are normally a powdery solid and that need to be blended with unsaturated or small chain lipids that are liquid at room temperature in order to achieve a waxy texture that will perform well as a coating stick. Saturated lipids that are normally a powder and need such modification include hydrogenated phospholipids and fully saturated triacylglycerols such as trimyristate, tristearate, or tripalmitate. The lipids can be fully mixed with either unsaturated and/or short chain lipids such as unsaturated medium-chain triacylglycerols from corn oil, olive oil, palm oil, sunflower oil, or rapeseed oil for instance, or unsaturated simple fatty acids (oleic acid, linoleic acid). The solvent based process can facilitate the mixing of these types of lipid components.

After packaging the formed product (e.g., a stick, which may be placed into an applicator as discussed below) and wrapping or boxing to maintain a sterile barrier, the product may be terminally sterilized by gamma irradiation or by electron beam sterilization. Alternatively, the product may be prepared and packaged by aseptic processing.

### Devices and Delivery Systems

The present technology provides infection-inhibiting delivery systems comprising a) an infection-inhibiting composition and b) an applicator containing the infection-inhibiting composition. The applicator preferably supports the composition during application and is operable to deploy the composition as material is transferred to the device surface. In some embodiments wherein the composition is in stick form, the applicator comprises a substantially cylindrical tube having an open end and an advancing mechanism at the end of the tube opposite to the open end, wherein the infection-inhibiting composition is contained within the tube, and the advancing mechanism is operable to move the composition along the axis of the tube so as to extend a surface of the infection-inhibiting composition at the open end of the tube.

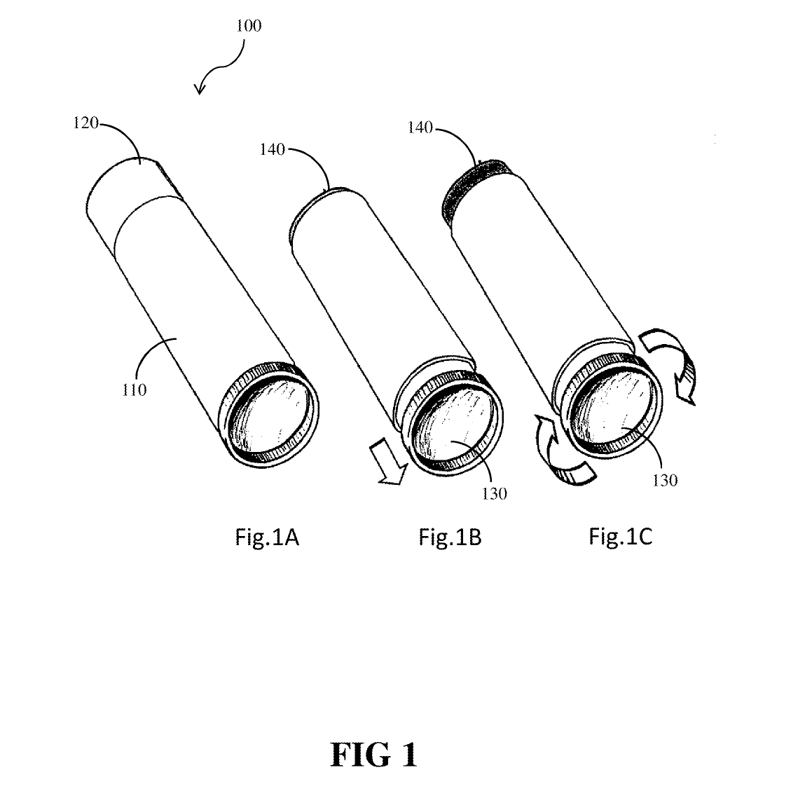

An exemplary infection-inhibiting delivery system 100 is shown in FIG. 1. The delivery system 100 comprises a housing 110, which is a substantially cylindrical tube, and an optional cap 120 for shielding the infection-inhibiting composition 140 when not in use. When in use, as shown in FIG. 1B, the base 130 is withdrawn from its secure connection with the housing, as depicted by the large white arrow, and the cap is removed to expose the infection-inhibiting composition 140. Then, as shown in FIG. 1C, the advancing mechanism 130 is rotated, as depicted by the curved white arrows, to move the infection-inhibiting composition 140 outwardly from the housing 110. Optionally, rotating the advancing mechanism 130 in the opposite direction urges the infection-inhibiting composition 140 back into the housing 110 until it is completely contained. At this time, the advancing mechanism 130 is then urged toward the housing 110 and snaps into the locked position where no rotation can take place. Such a dispensing device can be used to coat an orthopedic device with an infection-inhibiting composition of the present technology.

### Methods of Coating Implants

Methods for inhibiting the growth of microbes on a surface of a medical implant comprise depositing an infection-inhibiting material on the surface of an implant, by rubbing a composition comprising the infection-inhibiting material on the surface. Accordingly, the present technology provides methods for inhibiting infection at the site of implantation of an orthopedic device in a human or animal subject, comprising rubbing a surface of the device, prior to implantation, with an infection-inhibiting composition having a waxy matrix comprising an infection-inhibiting material selected from the group consisting of a lipid, an antimicrobial agent, and mixtures thereof, wherein a thin layer of the infection-inhibiting material is deposited on the surface of the device.

Preferably the implant is coated with the infection-inhibiting composition prior to implantation. In particular, the implant may be obtained from a manufacturer or supplier, and then coated with the infection-inhibiting composition at a time proximate to the time of implantation. (As used herein, a “proximate” time is any time 24 hours or less before implantation, 4 hours, 2 hours, 1 hour, 30 minutes, 15 minutes, 10 minutes, 2 minutes, 1 minute, or less, before implantation.) Such methods may be considered “intraoperative” wherein the coating is performed by a surgeon or other health care provider as part of the implantation surgery. Such intraoperative methods may offer advantages, such as allowing the health care professional to apply the infection-inhibiting coating in a location, manner, and quantity specifically adapted to the procedure and risks factors for surgical site infection as assessed during the surgery. In various embodiments, the compositions are applied to the surface of the implant in 10 minutes or less, preferably 5 minutes or less. In one embodiment, coating the implant intraoperatively comprises selecting an infection-inhibiting composition from a plurality of infection-inhibiting compositions, wherein each composition has a matrix different than the matrix of another composition of the plurality. Additionally, coating the implant intraoperatively may also comprise selecting an infection-inhibiting composition from a plurality of infection-inhibiting compositions, wherein each composition has an antimicrobial agent different from than the agent of another composition of the plurality.

In other embodiments, the implant is coated with the infection-inhibiting composition prior to implantation. For example, a cemented or uncemented implant can be coated with the infection-inhibiting composition during a manufacturing process, and the coated implanted can be sterilized and sealed in sterile packaging. Such methods may be considered “preoperative” wherein the coating is performed by a manufacturer while the implant is being fabricated. Preoperative coating can be performed by rubbing, spreading, smearing, or otherwise applying the infection-inhibiting composition onto a textured implant, wherein the applying is performed with an applicator. Alternatively, the preoperative coating can be performed by dipping a textured implant into the infection-inhibiting composition, or the infection-inhibiting composition can be sprayed onto the textured implant. Accordingly, in various embodiments, the implant is brush coated, spray coated, roll coated, printed, sputtered, or dip coated with the infection-inhibiting composition. During a procedure, such as a revision procedure, a medical professional can choose a preoperatively coated implant that is coated with a particular infection-inhibiting composition that is suitable for the infection being treated.

Implants used in the methods of the present technology include any implant that is at least partially implanted into the body of a subject. An orthopedic implant can include implants that span across the skin layers interfacing with an internal tissue, such as a hard tissue like bone, or a soft tissue like muscle or cartilage, or with another implant. Orthopedic implants useful in the present technology can also include prosthesis parts and accessory components interfacing such prosthesis parts. Generally, the surfaces of the implant are completely or partially implanted into the body of the subject, comprising a metal substrate having one or more surfaces operable to contact a bone tissue or soft tissue when implanted. Orthopedic implants useful in the present technology may be permanent tissue replacement devices, permanent stabilization devices, or temporary skeletal stabilization devices.

The orthopedic implants of the present technology include prosthetic implants or parts thereof. Joint replacement systems that may be coated include uncemented hips, knees, elbow, or shoulders. Orthopedic implants include uncemented devices that require tissue ingrowth or ongrowth to stabilize the implant, for example, for use in hip implants (e.g., femoral stems), knee implants (e.g., acetabular cups), elbow implants, shoulder implants, prosthetic frames, bone prostheses, and small joint prostheses. The devices to be coated can also include devices that do not require biologic fixation, such as fracture stabilization hardware (intramedullary nails, plates, screws), and arthrodesis hardware. Internal and external fixation implants and devices include bone plates, anchors, bone screws, rods, intramedullary nails, arthrodesis nails, pins, wires, spacers, and cages. The coating could also be applied to transdermal devices such as external fixation pins used in fracture stabilization or limb lengthening procedures. Such devices are commercially available from leading orthopedic device manufacturers, including Biomet Inc. (Warsaw, Ind., USA). Other manufacturers include Zimmer, Inc. (Warsaw, Ind., USA); DePuy Orthopedics, Inc. (Warsaw, Ind., USA) and DePuy Spine, Inc. (Raynham, Mass., USA).

The orthopedic implants of the present technology can comprise solid metals, for example, gold, silver, stainless steel, platinum, palladium, iridium, iron, nickel, copper, titanium, aluminum, chromium, cobalt, molybdenum, vanadium, tantalum, and alloys thereof. In preferred embodiments, the orthopedic implant comprises a metal including surgical stainless steel, titanium or a titanium alloy. In yet other embodiments, the orthopedic implant comprises a polymer, such as polyethylene, or a ceramic.

One or more surfaces of the implant, for example the surface to be coated with the infection-inhibiting coating, may be textured. The textured surfaces enable an inhibitory amount of biodegradable coating comprising an antimicrobial agent to be applied to the implant, and after the coating is degraded, the textured surfaces promote bone ingrowth. As used herein, an “inhibitory amount” of the coating is an amount sufficient to treat an infection or inhibit an infection from forming. Accordingly, in various embodiments, the implants can be implanted in subjects without cement. The orthopedic implant surface to be coated with an infection-inhibiting coating can be textured uniformly with surface irregularities, including pores (micropores), dimples, spikes, ridges, grooves (e.g., microgrooves), roughened texture (e.g., microtextured), surface grain, strips, ribs, channels, ruts. The size of the micropores, dimples, spikes, ridges, grooves (e.g., microgrooves), roughened texture (e.g., microtextured), surface grain, strips, ribs, channels, ruts can range from about 1 μm to about 2000 μm. In some embodiments, the size ranges from about 10 μm to about 100 μm. In another embodiment, implant surface has pores from about 200 μm to about 2000 μm. The porosity is enough to retain a sufficient quantity of the infection-inhibiting coating to treat or inhibit infection or reinfection and to promote bone in-growth. The texture may be formed by any suitable methods, for example, by molding, chemical etching, roughening with sandpaper or other abrasives (e.g., sand blasting and glass bead blasting), electrical means (such as EDM machining), thermal means, laser etching, or additive manufacturing processes. Roughed surfaces include porous plasma sprayed titanium “porous plasma spray,” sintered beads, and sintered wire meshes. Especially with the use of additive manufacturing processes, implants can be customized to match a subject's unique anatomy. In other words, additive manufacturing allows for patient-specific devices.

Additive manufacturing processes utilize digital electronic file formats (e.g., STL files) that can be printed into three-dimensional (3D) CAD models, and then utilized by a prototyping machine's software to construct various implants based on the geometric orientation of the 3D model. The constructed implants are produced additively in a layer-wise fashion by dispensing a laser-fusible powder one layer at a time. The powder is fused, re-melted or sintered, by the application of laser energy that is directed in raster-scan fashion to portions of the powder layer corresponding to a cross section of the implant. After each layer of the powder is fused, an additional layer of powder is dispensed, and the process repeated, with fused portions or lateral layers fusing so as to fuse portions of previous laid layers until the implant is complete. Accordingly, a method for forming an implant having a porous region comprises imaging bone at an infection site with a high resolution digital scanner, such as a computed tomography (CT) scanner or other 3-dimensional scanner, to generate a three-dimensional design model of the bone; removing a three-dimensional section form the design model; fabricating a porous region on a digital representation of the implant by replacing a solid portion of the digital implant with the section removed from the digital representation; and using an additive manufacture technique to create a physical implant including the fabricated porous region. In various embodiments, the additive manufacture technique comprises a Direct Metal Laser Sintering (DMLS) process, an Electron Beam Melting (EBM) process, Selective Laser Sintering (SLS), Fused Deposition Modeling (FDM), Stereolithography (SLA), Laminated Object Manufacturing, Powder Bed and Inkjet Head 3D Printing and Plaster-Based 3D Printing (PP). Various methods for making textured implants and for coating implants are provided in U.S. Pat. No. 8,388,887, Gupta et al., issued Mar. 5, 2013; U.S. Patent Application Publication No. 2014/0025181, Vanasse et al., published Jan. 23, 2014; and U.S. Patent Application Publication No. 2013/0204384, Hensley et al., published Aug. 8, 2013.

In accordance with the present technology, the orthopedic implants can be coated with infection-inhibiting composition on at least one surface of the implant. Various methods for coating implants are described in U.S. Patent Application Publication No. 2011/0143127, Gupta et al., published Jun. 16, 2011. Accordingly, the present technology provides methods for making an implantable medical device having an infection-inhibiting coating, the methods comprising rubbing an infection-inhibiting composition on a surface of the medical device, wherein a thin layer of the infection-inhibiting composition is coated on the surface, and wherein the infection-inhibiting composition comprises a waxy matrix comprising an infection-inhibiting material selected from the group consisting of a lipid, an antimicrobial agent, and mixtures thereof, wherein the waxy carrier is operable to deposit the infection-inhibiting material when rubbed on a surface of the device. In various embodiments, the device is coated with the infection-inhibiting composition by brush coating, spray coating, roll coating, printing, sputtering, or dip coating.

In some embodiments, all surfaces of the implant exposed to body tissues are coated. In other embodiments, the surfaces of the orthopedic implant to be coated with an infection-inhibiting composition are surfaces that are not intended to provide a structural network for tissue or cellular ingrowth or ongrowth. In some embodiments for coating of implants having articulating surfaces (e.g., hip implants), the infection-inhibiting composition is coated on an articulating surface of the implant.

In some embodiments, the waxy matrix dissipates from the medical device immediately after implantation. In particular, for use on orthopedic implants that are stabilized by biologic fixation or integration with host tissue, the lipid composition preferably dissolves from the implant surface very rapidly so as not to interfere with bone tissue ingrowth or ongrowth. The matrix may dissipate from the surface of the coated implant within hours after implantation. In some embodiments, the anti-infective is preferably released from the carrier over time, such as over the course of from about 1 day to about 3 weeks, or from about 3 to about 10 days.

The infection-inhibiting compositions can be applied in any appropriate manner, including application methods known to those of ordinary skill in the art of coated medical devices. For example, the infection-inhibiting composition can be rubbed or wiped onto the orthopedic implant. As discussed above, the composition can be applied using an infection-inhibiting delivery system of the present technology.

### Methods for Treating Infected Joint Implants

Methods for treating infection at the site of implantation of an orthopedic device in a human or non-human animal subject comprise a revision with an implant coated with a composition comprising a waxy matrix and an infection-inhibiting material, as described above. Therefore, such methods comprise removing the orthopedic device from the subject, and implanting a replacement device, a surface of which is coated with an infection-inhibiting composition having a waxy matrix. The waxy matrix may comprise an infection-inhibiting material selected form the group consisting of a lipid, an antimicrobial agent, and mixtures thereof. In some embodiments, the method further comprises administering systemic antibiotics to the subject, such as by oral or intravenous administration.

In some embodiments, the antimicrobial agent is selected based on a diagnostic assessment of an infection. Therefore, an antimicrobial formulation can be “tailor made” for a specific patient. For example, a biopsy of an infected area can be obtained and the microorganisms contained therein can be cultured. The culture can then be screened to determine if the infecting organisms are sensitive to any particular antimicrobial agents. This information can be used to develop an antibiogram that reflects what organisms are present in the culture and to what antimicrobial agents the organisms are susceptible. Therefore, an antibiotic formulation, for example, can be made based on a prescription developed from an antibiogram of infecting microorganisms. In one embodiment, the identity of infecting microorganisms can be determined during a surgical procedure with a point of care diagnostic device, such as a biosensor. The point of care device can be any such device commonly used in the art. One device for performing a point of care diagnostic assessment is described in U.S. Patent Application Publication No. 2013/0230844, Egan et al, published Sep. 5, 2013.

Also as described above, at least one surface of the replacement device is textured with grooves, pore, divots, protrusions, or combinations thereof. For example, the surface can comprise a plurality of pores having a size of from about 200 μm to about 2000 μm. The texture allows for a sufficient amount of infection-inhibiting composition to be added to the replacement device to inhibit or treat an infection. Because the infection-inhibiting composition dissipates or biodegrades over time, the texture also allows for bone ingrowth into the replacement device. Therefore, the replacement device can be implanted without cement or antibiotic-loaded cement. Such devices may be referred to as uncemented devices. Use of uncemented devices may be beneficial in some methods because they can be more easily removed in the event of a subsequent infection.

The method for treating infection at the site of implantation of an orthopedic device can include one or two surgeries. In a two-stage revision, removing the orthopedic device is performed during a first surgery, which also includes implanting a temporary spacer comprising an antimicrobial agent. A second surgery comprises removing the temporary spacer and implanting the replacement device. In a one-stage revision, removing the orthopedic device, and implanting the replacement device is performed during a single surgery. A one-stage revision may also include debriding the implantation site after the orthopedic device is removed.

Although the coated implants of the current technology can be used in revision procedures to treat infection at the site of implantation of an orthopedic device, the coated implants can also be used during an initial implantation of a prosthetic device. When used initially, the coated implant can be used without cement. The infection-inhibiting material prevents or inhibits infections from forming near the site of the implant. When the infection-inhibiting material dissipates or biodegrades, a textured aspect of the implant, such as a plurality of pores, allows for bone ingrowth. This feature allows the implant to be implanted without cement or antibiotic-loaded cement. Therefore, if the site of implantation were to subsequently become infected, the uncemented implant could be removed easier than an implant that is cemented into place.

The materials and processes of the present technology are illustrated in the following non-limiting examples.

### Example 1

0.005 g of rifampin and 0.005 g of minocycline are dissolved in 0.05 g of ethanol to form an antimicrobial mixture. About half of the ethanol is allowed to evaporate to form a concentrated antimicrobial mixture, which is then stirred into 0.1 g of Phosal® 53 MCT (Lipoid Group, Köln, Germany) until a uniform antimicrobial and lecithin mixture is formed. The antimicrobial/lecithin mixture is then folded into 10 g of Phospholipon® 90G (Lipoid Group, Köln, Germany) until a uniform composition is formed. The final composition contains:

0.1% each of rifampin and minocycline;

0.5% ethanol;

1% of a mixture of 50% phosphatidylcholine and 50% various other lipids; and

98.4% of a mixture of at least 90% phosphatidyl choline and the remainder a mixture of various other smaller lipids.

### Example 2

Formulation “90G” was made consisting entirely of Phospholipon 90G purified soy phosphatidylcholine, with a minimum purity of 94% phosphatidylcholine by weight. The yellowish, waxy solid material is supplied as small clumps. To form the material into a stick-form composition, it was repeatedly ground in a ceramic mortar and pestle that was heated to 40° C., then kneaded until solid, and then 4 gram aliquots were cold pressed into a 12 mm diameter cylinder.

### Example 3

Formulation “90G90H” was made by grinding together 6 grams of Phospholipon 90G and 3 grams of Phospholipon 90H (Lipoid Group, Köln, Germany). Phospholipon 90H is white powder purified soy derived phosphatidylcholine that is hydrogenated (fully saturated). The Phospholipon 90H was blended with the unsaturated natural phosphatidylcholine, and preheated to 60° C. to soften the hydrogenated form. The two materials were finely ground together in a ceramic mortar and pestle that was heated to 40° C. The mixture was kneaded until a smooth, solid, and cohesive waxy solid was created. Four gram aliquots were cold molded into 12 mm diameter cylindrical sticks.

### Example 4

Formulation “90G53MCT” was made by combining 3 grams of Phospholipon 90G with 1 gram of Phosal 53MCT (Lipoid GmbH, Köln, Germany). Phosal 53MCT is a mixture of lipids, comprising at least 53% phosphatidylcholine, dissolved in medium chain triglycerides and sunflower oil. Phosal 53MCT is liquid at room temperature. A mixture of Phosal 53MCT and Phospholipon 90G was made by finely grinding together in a ceramic mortar and pestle that was heated to 40° C., then kneading until a smooth, solid, and cohesive waxy is solid formed. The waxy solid was cold molded into a 12 mm diameter cylindrical stick.

### Example 5

Formulation “10% R90G” was made by grinding together 0.8 grams of the antibiotic rifampin (Lupin Pharmaceuticals, Mumbai, India), and 7.2 grams of Phospholipon 90G in a ceramic mortar and pestle that was heated to 40° C., then kneading until a smooth, solid, and cohesive waxy solid is formed. 4 gram aliquots were cold molded into 12 mm diameter cylindrical sticks.

### Example 6

Formulation “25% R90G” was made by combining 2 grams rifampin and 6 grams Phospholipon 90G and grinding, kneading, and molding into cylindrical sticks as per Example 5, above.

### Example 7

Formulation “10% R90G90H” was made by grinding together 0.8 grams of the antibiotic rifampin (Lupin Pharmaceuticals, Mumbai, India), and 7.2 grams of the 90G90H carrier from formulation “90G90H” in a ceramic mortar and pestle that was heated to 40° C., then kneaded until a smooth, solid, and cohesive waxy solid is formed. 4 gram aliquots were cold molded into 12 mm diameter cylindrical sticks.

### Example 8

Formulation “25% R90G90H” was made by combining 2 grams rifampin and 6 grams of 90G90H and mixing and molding into cylindrical sticks as in Example 7, above.

### Example 9

Formulation “10% R90G53MCT” was made by mixing together 0.8 g rifampin and 1.8 grams of Phosal 53MCT, then grinding together with 5.4 grams of Phospholipon 90G to form a mixture. The mixture was finely ground in a ceramic mortar and pestle that is heated to 40° C., and then kneaded until a smooth, solid, and cohesive waxy solid was formed. 4 gram aliquots of the waxy solid were then cold molded into 12 mm diameter cylindrical sticks.

### Example 10

Formulation “10% V90G” was made by grinding a 0.8 grams of the antibiotic vancomycin (Axellia Pharmaceuticals, Oslo, Norway), and 7.2 grams of Phospholipon 90G in a ceramic mortar and pestle that was heated to 40° C. The mixture was then kneaded until a smooth, solid, and cohesive waxy solid formed. 4 gram aliquots of the waxy solid were then cold molded into 12 mm diameter cylindrical sticks.

### Example 11

Formulation “25% V90G” was made by combining 2 grams vancomycin and 6 grams Phospholipon 90G and grinding, kneading, and molding into cylindrical sticks in Example 10, above.

### Example 12

Formulation “10% PLY90G” was made by grinding 0.8 grams of the food preservative, epsilon polylysine (Zhengahou Bainfo Bioengineering company, China) and 7.2 grams of Phospholipon 90G in a ceramic mortar and pestle that is heated to 40° C. The mixture was kneaded until a smooth, solid, and cohesive waxy solid was formed. 4 gram aliquots of the waxy solid were cold molded into 12 mm diameter cylindrical sticks.

### Example 13

Gentamicin sulfate powder was mixed with Phospholipon 90G by finely grinding in a ceramic mortar and pestle. The mixture was kneaded until a smooth, uniform, cohesive solid was obtained. The solid was then molded into 12 mm diameter cylindrical sticks. Two formulations were mixed and molded: 10% gentamicin sulfate by weight, and 25% gentamicin sulfate by weight.

### Testing

The compositions of Examples 2-12, summarized in Table 1 below, were tested for hardness and density of coating when applied to a surface, and coating performance.

TABLE 1
Summary of Composition Formulations by weight percent

| | Carrier lipids | | | | | |
|---|---|---|---|---|---|---|
| | Purified soy | Hydrogenated | | | | |
| | phosphatidyl- | phosphatidyl- | | | | |
| | choline | choline | | Antimicrobials | | |
| | Phospholipon | Phospholipon | Phosal | | | Epsilon |
| Formulation name | 90G | 90H | 53MCT | Rifampin | Vancomycin | polylysine |
| 90G | 100 | | | | | |
| 90G90H | 67 | 33 | | | | |
| 90G53MCT | 75 | | 25 | | | |
| 10% R90G | 90 | | | 10 | | |
| 25% R90G | 75 | | | 25 | | |
| 10% R90G90H | 60 | 30 | | 10 | | |
| 25% R90G90H | 50 | 25 | | 25 | | |
| 10% R90G53MCT | 67.5 | | 22.5 | 10 | | |
| 10% V90G | 90 | | | | 10 | |
| 25% V90G | 75 | | | | 25 | |
| 10% PLY90G | 90 | | | | | 10 |

### Mechanical Testing

12 mm diameter unconstrained cylinders 12 mm tall of each of the 11 formulations of Examples 2-12 were tested for hardness by cone penetration, using a cone having an about a 27.5 degree angle that widened to a 6.25 mm diameter over a 6 mm distance. The cone was lowered into the 12 mm diameter 12 mm tall cylinder of the composition material at a rate of 1 mm per second for a distance of 5 mm. A ten pound load cell was used to measure the hardness as the peak load experienced by the cone over the 5 mm distance of travel into the sample. This test is a modification of the ASTM standard test method D1321 for needle penetration of petroleum waxes. The results of the mechanical testing are shown in Table 2. All the formulations had a hardness of between 1.9 to 10.2 lbf.

TABLE 2
Mechanical testing results

| Formulation name | Hardness (lbf) |
|---|---|
| 90G | 3.4 |
| 90G90H | 7.9 |
| 90G53MCT | 1.9 |
| 10% R90G | 3.6 |
| 25% R90G | 6.3 |
| 10% R90G90H | 4.9 |
| 25% R90G90H | 5.6 |
| 10% R90G53MCT | 2.2 |
| 10% V90G | 8.2 |
| 25% V90G | 10.2 |
| 10% PLY90G | 5.9 |

### Coating Density and Antimicrobial Dose

The infection-inhibiting compositions of Examples 2-12 were rubbed onto tared coupons of both smooth-finished and rough-finished (30-grit blasted) titanium alloy. The weight of the coating that transferred was recorded for each formulation. An aqueous saline solution was tested in lieu of a concentrated solution of antibiotic. Typical concentration of an antibiotic solution for IV use (prior to further dilution in the injection solution) was 50 mg per ml of water. The amount of antibiotic per cm² of surface area was calculated based on the weight of the coating and the concentration in the infection-inhibiting composition, or for the antibiotic solution, was approximated by the volume of water that adhered to the surface. Table 3 shows the coating densities applied onto the coupons.

TABLE 3
Coating Densities Applied to Coupons

| | Coating | Coating | Antimicrobial | Antimicrobial |
|---|---|---|---|---|
| | Weight on | Weight on | Agent Dose | Agent Dose |
| | Smooth | Rough | on Smooth | on Rough |
| Formulation | Surface | Surface | Surface | Surface |
| name | (mg/cm²) | (mg/cm²) | (μg/cm²) | (μg/cm²) |
| 90G | 0.99 | 2.9 | — | — |
| 90G90H | 0.28 | 1.7 | — | — |
| 90G53MCT | 0.28 | 3.2 | — | — |
| 10% R90G | 0.21 | 2.3 | 21 | 230 |
| 25% R90G | 0.18 | 1.8 | 44 | 450 |
| 10% R90G90H | 0.11 | 1.9 | 11 | 190 |
| 25% R90G90H | 0.14 | 1.7 | 35 | 425 |
| 10% | 0.42 | 3 | 42 | 300 |
| R90G53MCT | | | | |
| 10% V90G | 0.60 | 2.2 | 60 | 220 |
| 25% V90G | 0.25 | 4.1 | 62 | 1025 |
| 10% PLY90G | 0.46 | 1.9 | 46 | 190 |
| Antibiotic | 0.04 | 1 | 2 | 50 |
| Solution | | | | |

The embodiments and the examples described herein are exemplary and not intended to be limiting in describing the full scope of compositions and methods of the present technology. Equivalent changes, modifications and variations of embodiments, materials, compositions and methods can be made within the scope of the present technology, with substantially similar results.

### Non-Limiting Discussion of Terminology

The headings (such as “Introduction” and “Summary”) and sub-headings used herein are intended only for general organization of topics within the present technology, and are not intended to limit the disclosure of the present technology or any aspect thereof. In particular, subject matter disclosed in the “Introduction” may include novel technology and may not constitute a recitation of prior art. Subject matter disclosed in the “Summary” is not an exhaustive or complete disclosure of the entire scope of the technology or any embodiments thereof. Classification or discussion of a material within a section of this specification as having a particular utility is made for convenience, and no inference should be drawn that the material must necessarily or solely function in accordance with its classification herein when it is used in any given composition.

The citation of references herein does not constitute an admission that those references are prior art or have any relevance to the patentability of the technology disclosed herein. Any discussion of the content of references cited in the Introduction is intended merely to provide a general summary of assertions made by the authors of the references, and does not constitute an admission as to the accuracy of the content of such references. All references cited in the “Description” section of this specification are hereby incorporated by reference in their entirety.

The description and specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific examples are provided for illustrative purposes of how to make and use the compositions and methods of this technology and, unless explicitly stated otherwise, are not intended to be a representation that given embodiments of this technology have, or have not, been made or tested.

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

As used herein, the term “operable” refers to a material, device or action which is capable, by virtue of its composition, design or features, to perform a recited function. In some embodiments, an operable material device or action is adapted to perform the function, having a specific composition, design or feature that is adapted (relative to similar composition, design or features known in the art), individually or in combination with other composition, design and features of the present technology, for use in performing the recited function. An operable material, device or action may, in some embodiments, also be capable of performing other functions.

Disclosure of values and ranges of values for specific parameters (such as temperatures, molecular weights, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, and 3-9.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting ingredients, components or process steps, Applicants specifically envision embodiments consisting of, or consisting essentially of, such ingredients, components or processes excluding additional ingredients, components or processes (for consisting of) and excluding additional ingredients, components or processes affecting the novel properties of the embodiment (for consisting essentially of), even though such additional ingredients, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

1. A method for treating infection at a site of implantation of an orthopedic device in a human or animal subject, comprising
(a) removing the orthopedic device; and
(b) implanting a replacement device, a surface of which is coated with an infection-inhibiting composition having a waxy matrix, the waxy matrix comprising an infection-inhibiting material selected from the group consisting of a lipid, an antimicrobial agent, and mixtures thereof.

2. The method for treating infection according to claim 1, wherein the waxy matrix comprises a lipid selected from the group consisting of fatty acids, triacylglycerols, diacylglycerols, glycerophospholipids, and mixtures thereof.

3. The method for treating infection according to claim 2, wherein the waxy matrix comprises about 90% or more, by weight of the waxy matrix, of phosphatidylcholine.

4. The method for treating infection according to claim 1, wherein the infection-inhibiting composition comprises an antimicrobial agent selected from the group consisting of antibiotics, antimicrobial peptides, synthetic mimics of antimicrobial peptides, disinfectants, antimicrobial metal ions, epsilon polylysine, sugar alcohols, essential oils, and mixtures thereof.

5. The method for treating infection according to claim 4, wherein the antimicrobial agent is selected from the group consisting of fosfomycin, rifampin, tetracyclines, vancomycin, gentamycin, aminoglycosides, quinolones, glycopeptides, and mixtures thereof.

6-7. (canceled)

8. The method for treating infection according to claim 4, wherein the antimicrobial agent is present at a level of from about 0.1% to about 35% by weight.

9. The method for treating infection according to claim 1, wherein the infection-inhibiting composition has a cone penetration test hardness of at least 1.5 lbf.

10-11. (canceled)

12. The method for treating infection according to claim 1, wherein the infection-inhibiting composition is applied to the surface of the replacement device using an applicator comprising a substantially cylindrical tube having an open end and an advancing mechanism at the end of the tube opposite to the open end, wherein the infection-inhibiting composition is contained within the tube, and the advancing mechanism is operable to move the composition along the axis of the tube so as to extend a surface of the infection-inhibiting composition at the open end of the tube.

13. The method for treating infection according to claim 1, wherein the replacement implant device is textured.

14. The method for treating infection according to claim 13, wherein the texture is formed by a process selected from the group consisting of molding, electrical deposition, thermal means, laser etching, additive manufacturing processes and combinations thereof.

15. The method for treating infection according to claim 13, wherein the texture of the replacement device comprises at least one of grooves, pores, divots, and protrusions.

16. The method for treating infection according to claim 1, wherein the surface of the replacement device comprises pores having a size of from about 200 μm to about 2000 μm.

17. (canceled)

18. The method for treating infection according to claim 1, wherein the replacement device is coated with the infection-inhibiting composition in the operating room prior to implantation in the human or animal subject.

19. The method for treating infection according to claim 1, wherein the removing and implanting are performed in a single surgical procedure.

20-22. (canceled)

23. A method for treating infection at a site of implantation of an orthopedic device in a human or animal subject, comprising
(a) removing the orthopedic device;
(b) rubbing a surface of a replacement device with an infection-inhibiting composition having a waxy matrix, the waxy matrix comprising an infection-inhibiting material selected from the group consisting of a lipid, an antimicrobial agent, and mixtures thereof; and
(c) implanting the replacement device.

24. The method for treating infection according to claim 23, wherein the removing and implanting are performed in a single surgical procedure.

25. (canceled)

26. The method for treating infection according to claim 23, wherein the method further comprises administering systemic antibiotics to the human or animal subject.

27-33. (canceled)

34. The method for treating infection according to claim 23, wherein the rubbing comprises selecting the infection-inhibiting composition from a plurality of infection-inhibiting compositions, wherein each infection-inhibiting composition has a matrix different than the matrix of another infection-inhibiting composition of the plurality.

35. (canceled)

36. A method for treating infection at a site of implantation of an implanted orthopedic device in a human or animal subject, comprising
(a) obtaining a replacement device having a textured surface coated with an infection-inhibiting composition having a waxy matrix, the waxy matrix comprising a lipid, an antimicrobial agent, and mixtures thereof, wherein the antimicrobial agent is selected based on a diagnostic assessment of the infection;
(b) removing the implanted orthopedic device; and
(c) implanting the replacement device.

37-38. (canceled)

39. The method for treating an infection according to claim 36, wherein the replacement device is a patient-specific device.

40-50. (canceled)
